(19) United States
(12) Patent Application Publication
(10) Pub. No.: US 2026/0216479 A1
(43) Pub. Date: Jul. 30, 2026

(54) MULTIFUNCTIONAL MEDICAL DEVICE WITH INTEGRATED DILATION BALLOON

(71) Applicant: EUSTAKIUS S.R.L., Senna Comasco (Como) (IT)

(72) Inventor: Lino DI RIENZO BUSINCO, Senna Comasco (Como) (IT)

(21) Appl. No.: 19/159,100

(22) PCT Filed: Mar. 19, 2024
(86) PCT No.: PCT/IB2024/052642

§ 371 Date: Aug. 22, 2025

(30) Foreign Application Priority Data
Mar. 21, 2023 (IT) 102023000005337

(51) Int. Cl. A61M 25/01; A61M 25/00; A61M 25/10

(52) U.S. Cl. CPC A61M 25/0136; A61M 25/008; A61M 25/10181

(57) ABSTRACT

A multifunctional medical device has a grip connected to a guide stem, a dilation balloon slidingly inserted into the grip and the guide stem and not removable therefrom, elements for adjusting a curvature of the guide stem associated with the grip and/or the guide stem and not removable therefrom, elements for adjusting a position of the dilation balloon associated with the grip and not removable therefrom, and elements for inflating/deflating the dilation balloon associated with the grip. The multifunctional medical device is suitable for balloon dilation treatment of rhino-sinusitis and tubal stenosis with a minimally invasive technique, and has a series of appropriately integrated, space-saving elements so that a user holds the whole assembly in one hand, and with thumb and forefinger operates movement and control elements.

The present invention relates to a multifunctional device with an integrated dilation balloon.

Such multifunctional device is particularly advantageous, for example, for the balloon dilation treatment of rhino-sinusitis and tubal stenosis with a minimally invasive technique.

To consider sinusitis only a condition of sinus inflammation is reductive. It is preferable to speak of rhino-sinusitis, as the inflammation always accompanies both the mucosa of the nose and that of the facial sinuses. The cause of sinusitis lies in the obstruction of the thin connection channel between the nasal fossa and the paranasal sinus. The curative approach must, now more than ever, be minimally invasive and as conservative as possible. For the past several years, a new technology has been introduced for the treatment of chronic rhino-sinusitis with mucus stagnation within the paranasal sinuses. Such new technology, called balloon sinus dilation (BSD), is used to supplement or often replace classic FESS (Functional Endoscopic Sinus Surgery). Balloon dilation sinuplasty involves the use of a small balloon that is inflated inside the sinus involved in the inflammation for a variable time of between 6 and 12 seconds, thus allowing rapid drainage and healing of said sinusitis.

On the other hand, with regard to episodes of otitis, especially those associated with catarrhal overproduction, a malfunction of the Eustachian tube is very often recognized as the cause. Even slight changes in altitude, fast trains, airplanes, underwater activity, and temperature changes are the most common conditions that stress the auditory tube and may aggravate tubal dysfunction. The main symptoms are a feeling of pressure in the ear sometimes with tinnitus, stuffiness and muffling hearing, up to actual ear pain. The most concerning risks of tubal obstruction, caused by defective middle ear ventilation, are the development of chronic secretory catarrhal otitis, even cholesteatoma, and permanent hearing loss.

The otofunctional results that may be achieved with balloon tubal dilation treatment are noteworthy. The technique involves the introduction inside the lumen of the tube, under endoscopic vision in complete safety, of a small balloon that is inflated to the prescribed pressure for a variable time of between 40 and 120 seconds.

Examples of medical devices for balloon tubal dilation treatment are known from US 2021/401479A1 , US 2021/196930A1 , US 2020/178775A1 , US 2021/386274A1 , US 2018/117290A1 , and US 2020/187967A1 .

From the observation of current clinical practice and instrumentation currently in use, it was noted that in some medical fields, such as otorhinolaryngology, there would be a need to adopt new, minimally invasive surgical methods that take advantage of endoscopic technologies already in use in other fields. Therefore, there is a need to design and develop new specific medical devices based on the conformation of the entry site, which may create the basis for a new and improved practice, innovating the technique and significantly improving the outcome for the patient in the short and medium-long term, without disregarding said safety for the patient.

There is also a need for new medical devices that ensure ease of use for the practitioner while minimizing the learning curve.

The object of the present invention is to create a single, multifunctional instrument capable of treating maxillary, ethmoidal, frontal, and sphenoidal sinusitis, or rhinotitis with Eustachian tube dysfunction, without having to resort to the procurement and storage of numerous different devices as is the case with technologies currently on the market, and with a faster learning curve.

Such object is achieved by means of a multifunctional device with integrated balloon according to claim 1. Further features of this device are contained in the dependent claims.

The features and advantages of the multifunctional device according to the present invention will become apparent from the following description, given as a non-limiting example in accordance with the figures in the accompanying drawings, wherein:

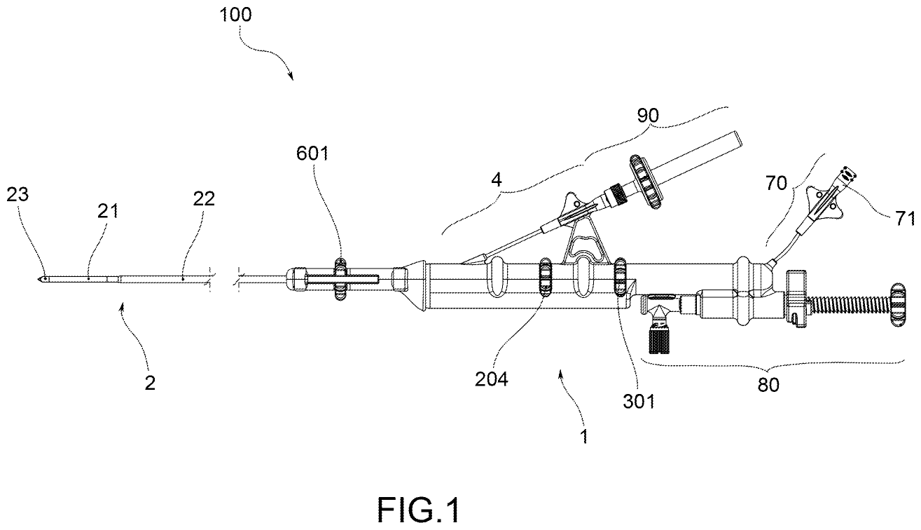

FIG. 1 shows a multifunctional device with an integrated dilation balloon according to the present invention in an embodiment example with a further associated device;

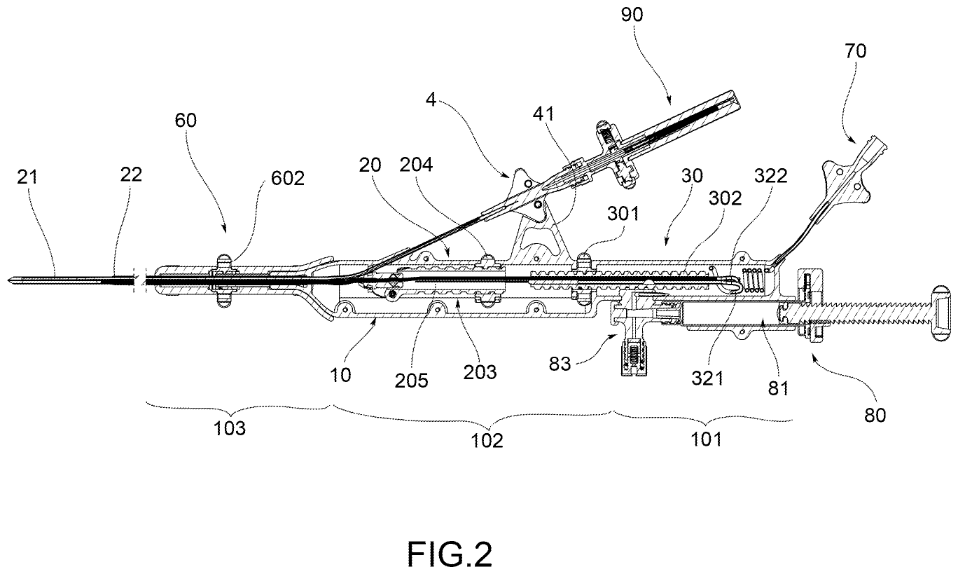

FIG. 2 shows the example from FIG. 1 in a sectional view;

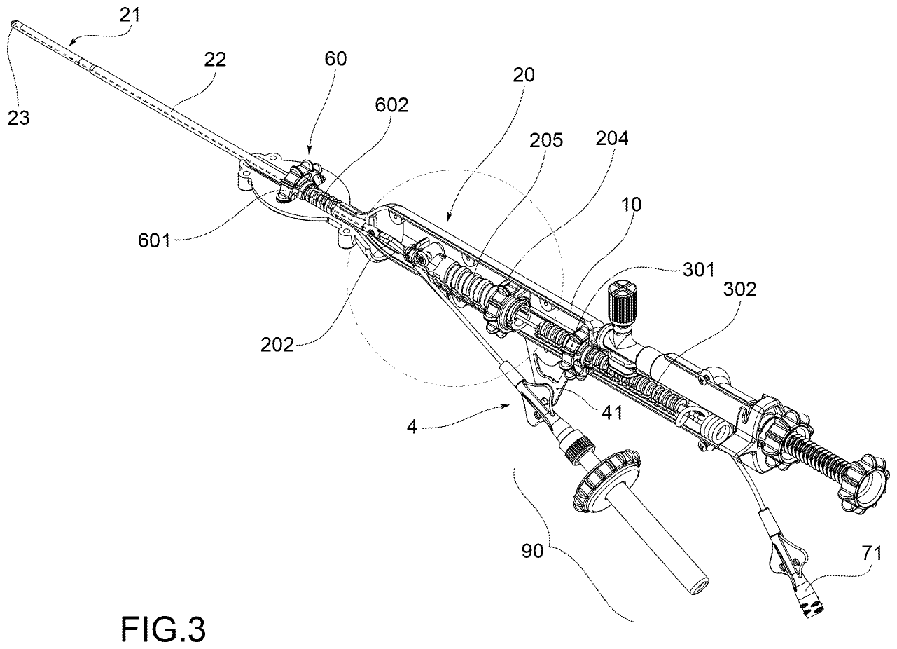

FIG. 3 shows the example from FIG. 1 in which a part of the shell of the grip has been removed to show the internal components;

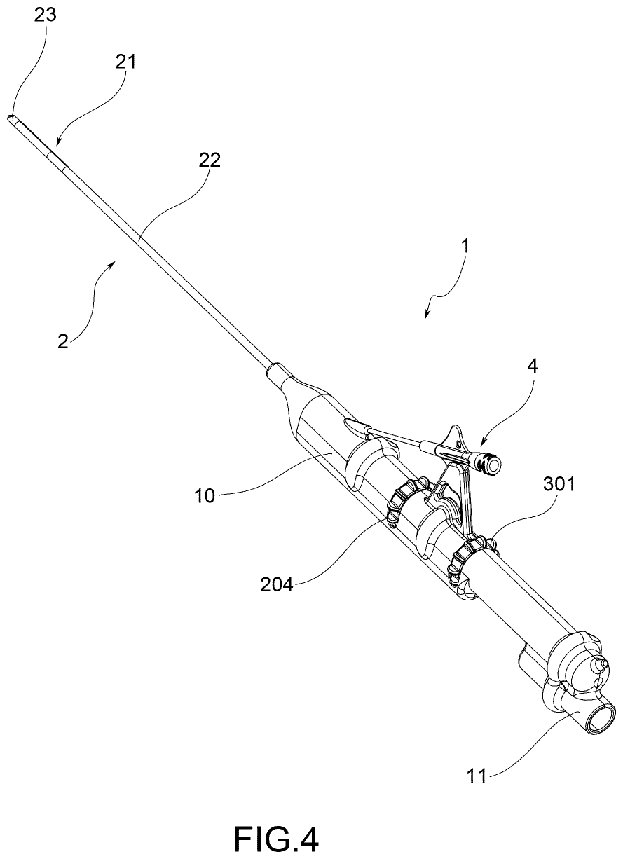

FIG. 4 shows only some components of the device, such as the grip;

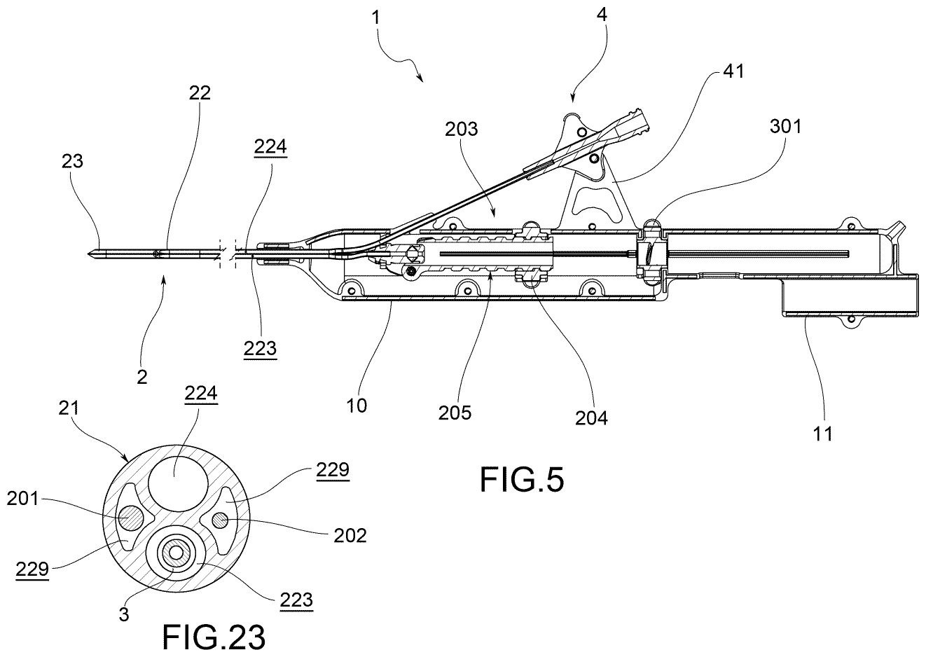

FIG. 5 shows a sectional view of FIG. 4;

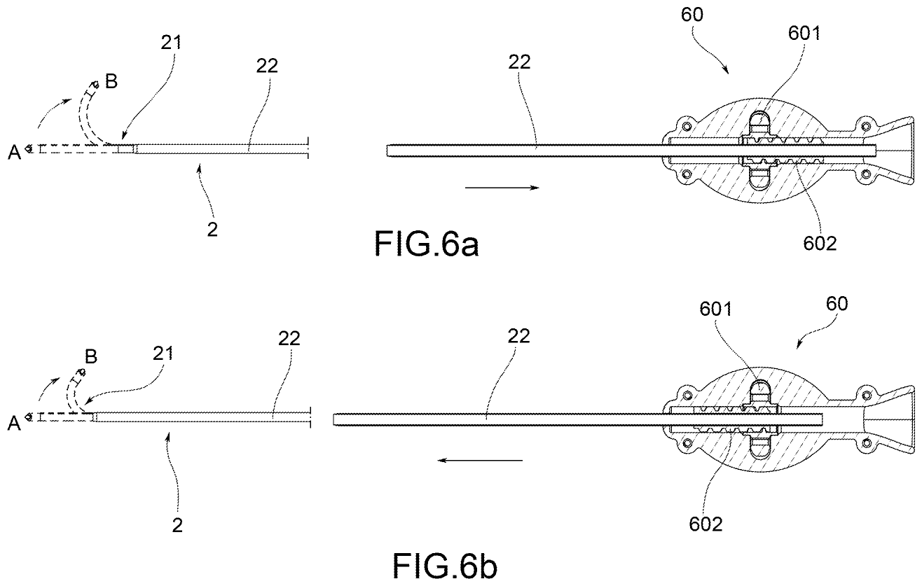

FIGS. 6a and 6b show the detail of the distal portion of the device in various configurations of curvature of the flexible end piece;

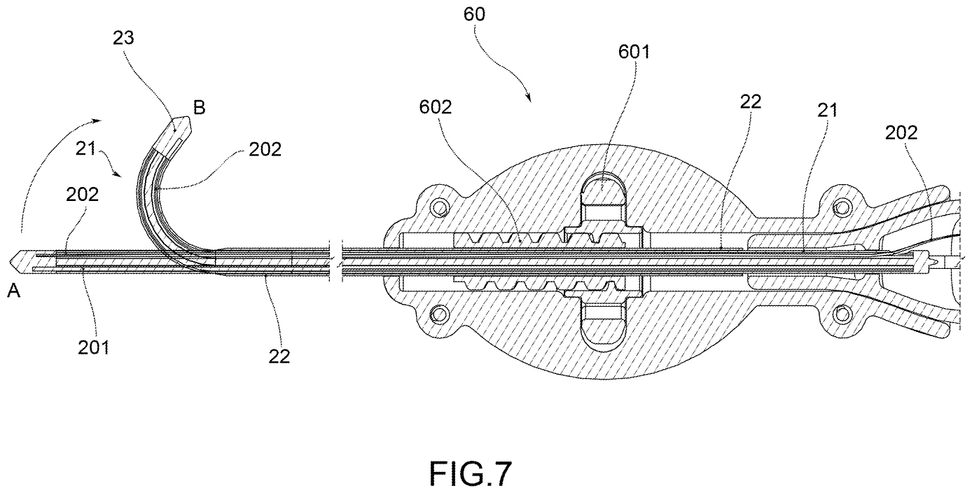

FIG. 7 shows a sectional view of the detail of the distal portion of the device, in various configurations of curvature of the flexible end piece;

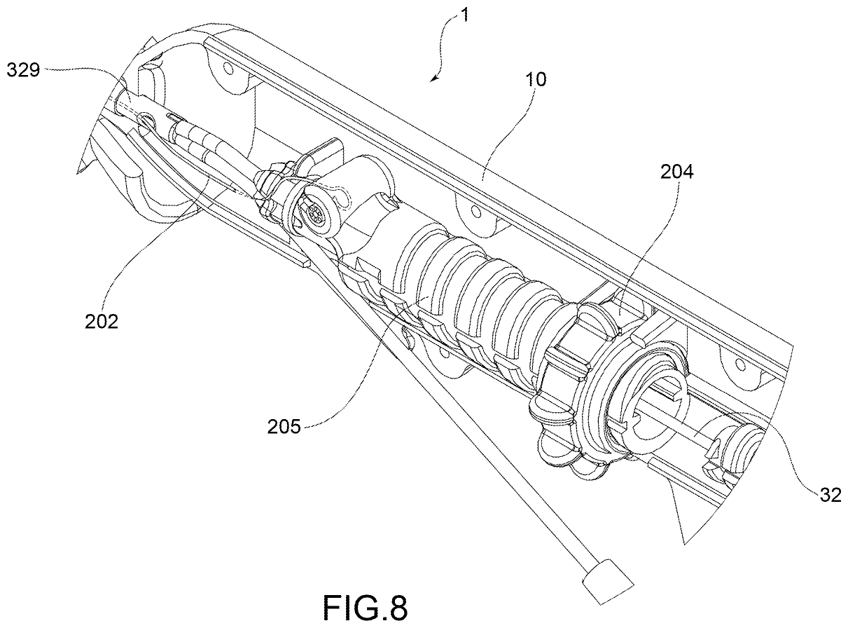

FIG. 8 shows a detail of FIG. 3, such as the primary curvature means of the guide stem;

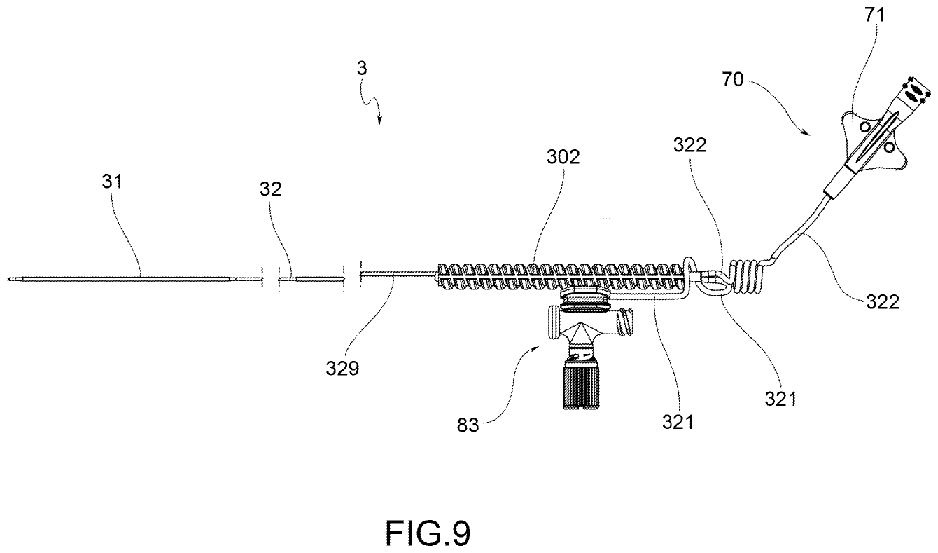

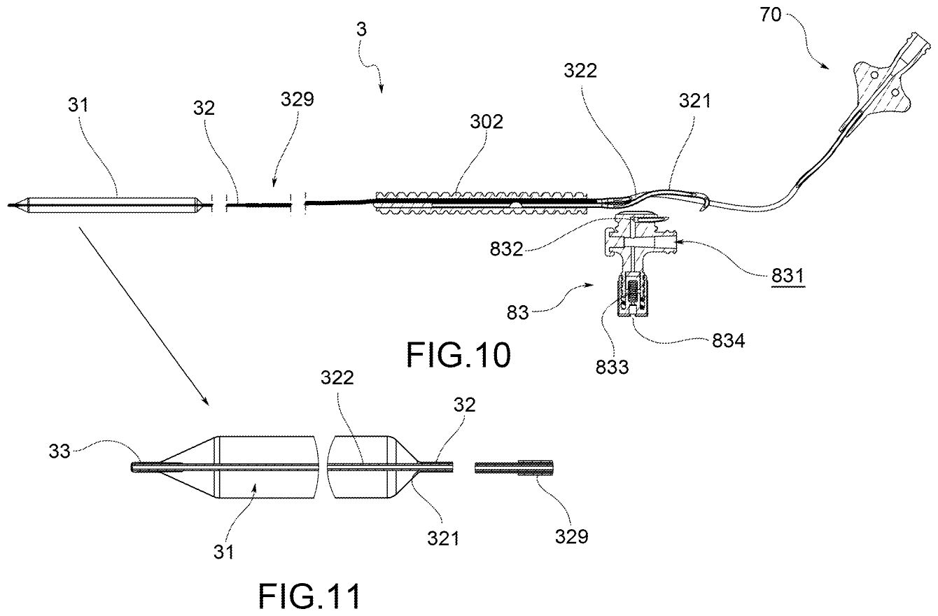

FIGS. 9 and 10 show only some components of the device, e.g., the dilation balloon and the associated components thereof, in deflated and expanded configurations, respectively;

FIG. 11 shows a detail of FIG. 10, for example the balloon;

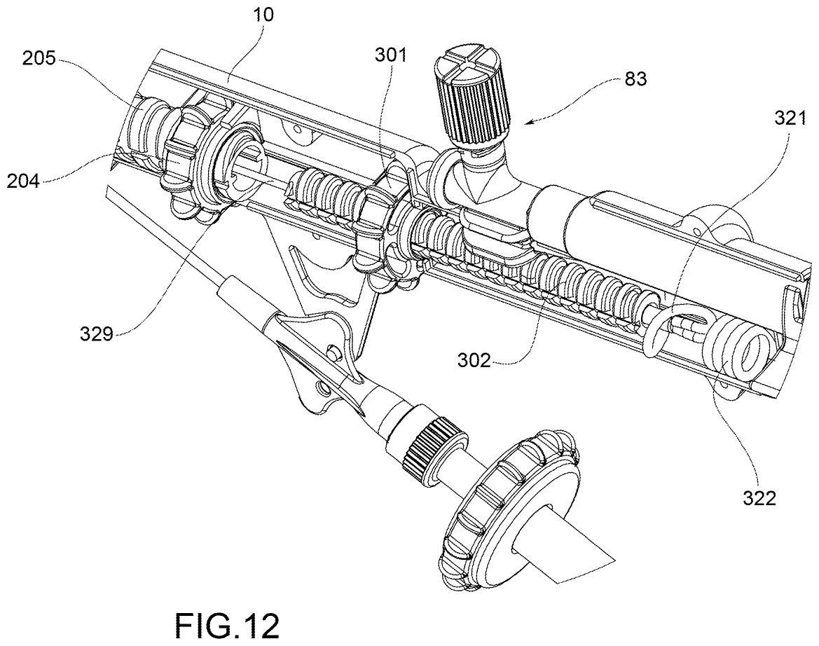

FIG. 12 shows a detail of FIG. 3, for example the means for adjusting the position of the dilation balloon;

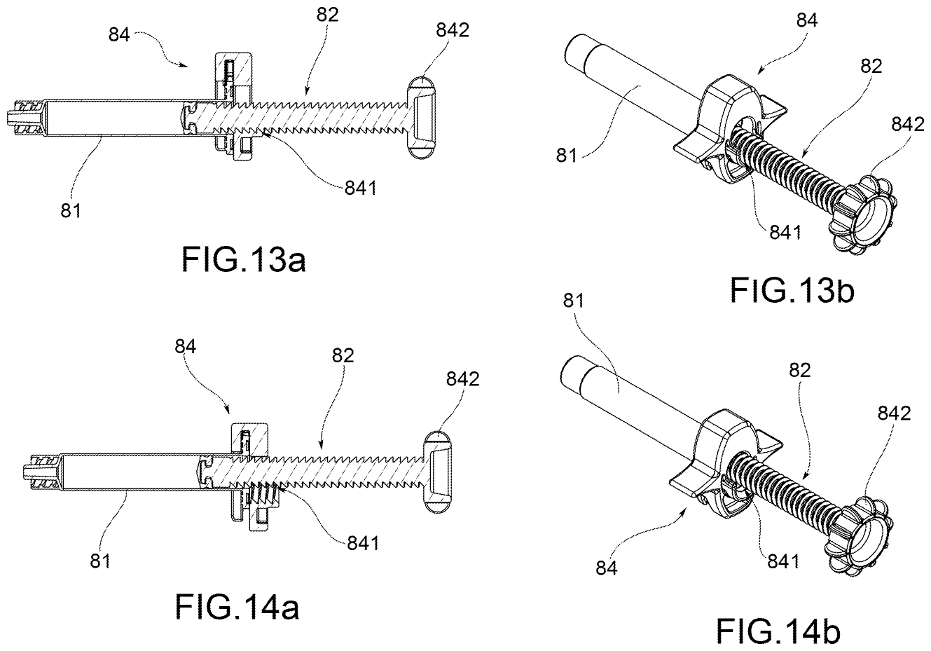

FIGS. 13a and 13b, 14a and 14b show some components of the inflation/deflation means of the dilation balloon, and in particular means for the controlled advancement of the piston of the inflation syringe, activated and deactivated, respectively;

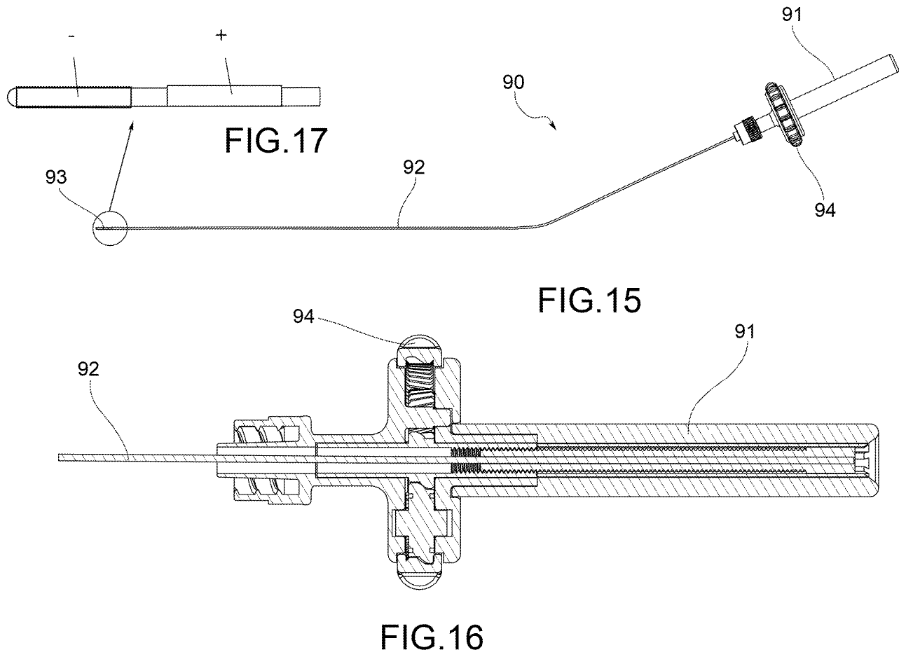

FIG. 15 to 17 show a further catheter, which may be associated with the multifunctional device, in its overall view (FIG. 15), in the detail of the knob 91 (FIG. 16), and in the detail of the accessories (FIG. 17);

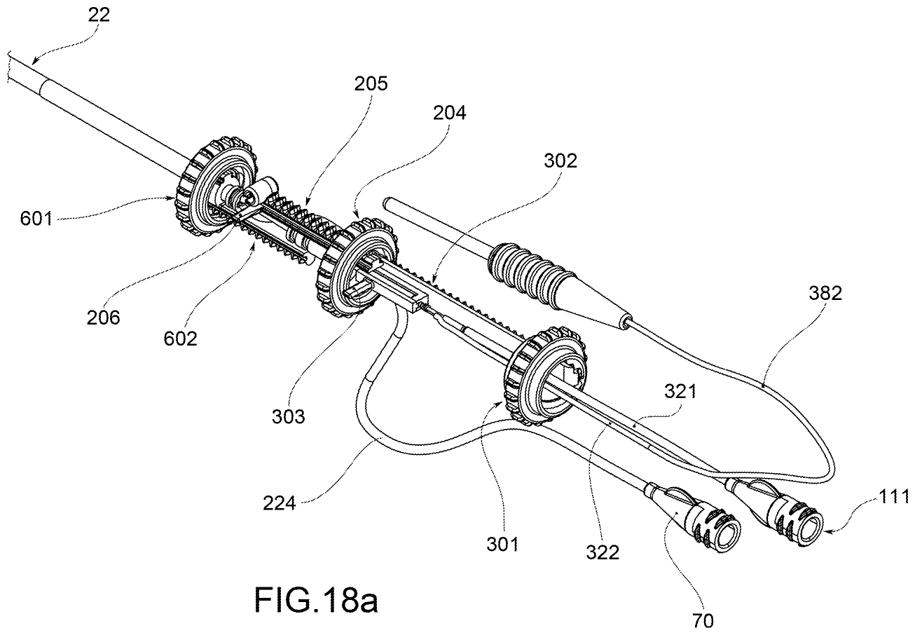

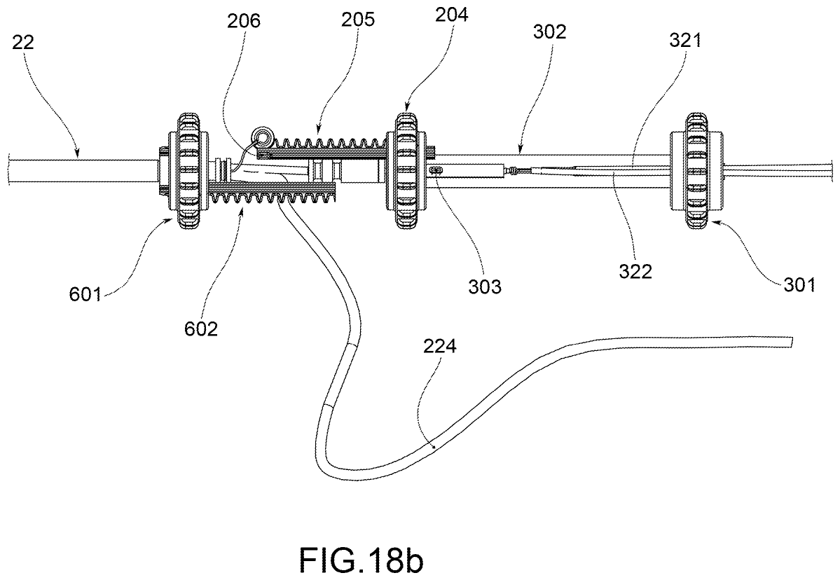

FIGS. 18a and 18b show a perspective view and a side view, respectively, of an embodiment variant of the multifunctional device that is the subject matter of the present invention, in which only a few internal components are shown, and in particular the primary operating means, the secondary curvature means, and the position adjustment means;

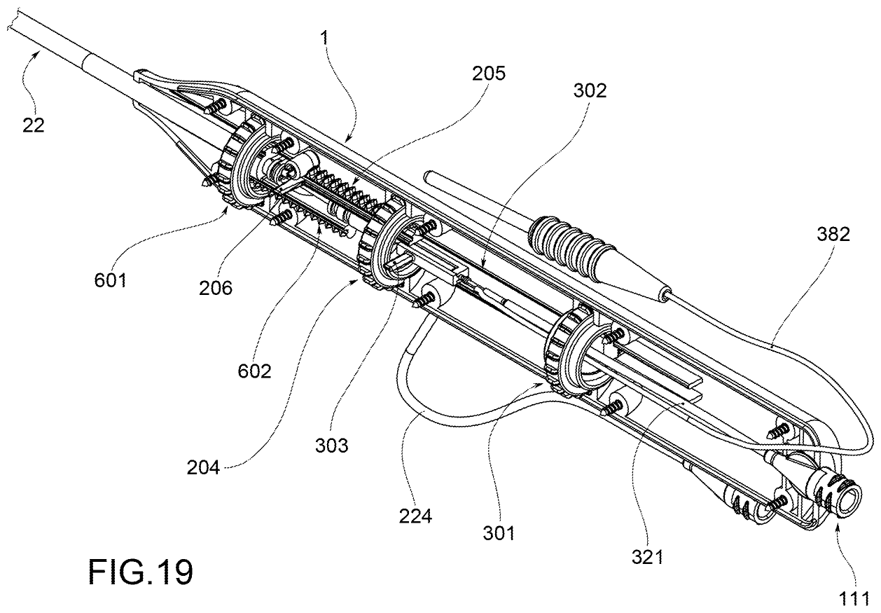

FIG. 19 shows the embodiment example from FIGS. 18a and 18b, with part of the shell of the grip added to represent the position of the various elements within said grip;

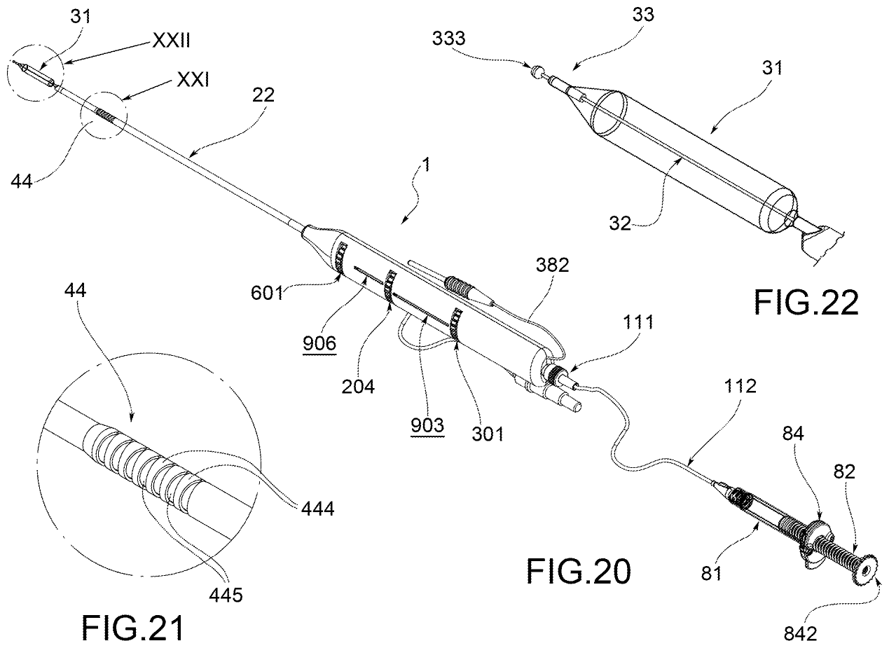

FIG. 20 shows an example of a multifunctional device with integrated, electrified dilation balloon with flexible section and syringe connection extension;

FIG. 21 shows a detail of the example in FIG. 20, in particular the flexible section of the outer body;
FIG. 22 shows a detail of the example in FIG. 20, in particular the area provided with the electrode and balloon;

FIG. 23 shows a section of the inner body tube 21.

With reference to the attached figures, reference number 100 has been used to indicate a multifunctional device with integrated dilation balloon according to the present invention.

The device 100 comprises a grip 1 connected to a guide stem 2 within which an integrated dilation balloon 3 slides. The device 100 further comprises a connector 4 for the insertion of further devices that may be used according to specific needs. The device 100 further comprises means for controlling the various components integrated in said multifunctional device, such as means for adjusting the curvature of the guide stem 2, means for adjusting the position of the dilation balloon 3, means for inflating/deflating the dilation balloon 3, and preferably also suction/injection means.

The grip 1 comprises a shell 10 that defines therein a cavity 12 in which various components are housed suitable for the movement and operation of the multifunctional device 100.

The grip 1 comprises a proximal portion 101, a central portion 102, and a distal portion 103.

The proximal portion 101 of the grip 1 has openings for the connection of the inflation/deflation means 80 of the dilation balloon 3 and any suction/injection means 70.

The central portion 102 of the grip 1 houses the primary curvature means 20 of the guide stem 2 and the position adjustment means 30 of the dilation balloon 3. The central portion 102 of the grip 1 also houses, fixed to an appropriate support 41, the connector 4 for the insertion of a further device 90. The distal portion 103 of the grip 1 houses secondary curvature means 60 of the guide stem 2.

The guide stem 2 is connected to the distal portion 103 of the grip 1.

The guide stem 2 comprises an inner body 21 fixed to the grip 1, on which an outer body 22 slides.

The distal end of the inner body 21, and thus of the guide stem 2, is a tip 23, preferably radiopaque.

As shown in FIG. 23, the inner body 21 is provided with two inner channels, or lumens, running parallel to each other along its entire length, including the tip 23.

A first inner channel 223 is intended to house the dilation balloon 3, sliding therein until it at least partially emerges from the tip 23.

A second inner channel 224 is connected to the connector 4 and is intended to house a further device 90, slidable therein until it emerges at least partially from the tip 23.

In an embodiment example, shown in FIG. 18a, the second inner channel 224 of the inner body 21 is connected to suction/injection means that enable indeed either fluid injection or suction by the tip 23. The suction/injection means comprise a connector 71 directly connected to the additional lumen 322 and integrated, i.e., non-removable therefrom, with the multifunctional device 100.

It should be noted that there may also be additional lumens, called service lumens 229.

The inner body 21 is flexible, at least in a distal portion thereof.

The guide stem 2, and in particular the distal portion of the inner body 21, is capable of switching from a straight configuration A to a curved configuration B, as seen in FIG. 7. The transition between the straight configuration A and the curved configuration B of the guide stem 2 is controlled by primary curvature means 20.

The primary curvature means 20 comprise:
at least one tie rod 202, housed internally in the inner body 21, which allows for the transition to the curved configuration B of the distal portion of the inner body 21;
primary operating means 203, associated with the grip 1, connected to the tie rod 202, suitable for controlling the advancement and retraction of the tie rod 202 to adjust the level of curvature.

In an embodiment example, there is provided a tie rod 202, which allows an upward curvature, and a straightening element in the form of a spindle 201, housed within the inner body 21, for example made of superelastic metal, which allows a return to the straight configuration A. Both the tie rod 202 and the spindle 201 are housed in service lumens 229 arranged in the inner body 21 of guide stem 2, as seen in FIG. 23.

In an alternative embodiment example, two opposing tie rods 202 are provided, housed within the inner body 21: the first tie rod is suitable for allowing an upward curvature and the second tie rod is suitable for allowing a downward curvature. In such embodiment example, the straightening spindle 201 is absent, as it is not needed.

The primary operating means 203 comprise a primary wheel 204 connected to a primary worm screw 205, with which the tie rod 202 is integral. Depending to the direction of rotation of the primary wheel 204, it is possible to move the primary screw 205 longitudinally (forward and backward), which, by moving, acts on the tie rod 202 thus allowing for the curvature of the distal portion of the guide stem 2.

In this context, the expression “worm screw” refers to a type of mechanism used to convert a rotary motion into a translatory linear motion.

In an embodiment shown for example in FIGS. 3 and 12, the primary screw 205 is a notched cylinder, and is engaged internally with the primary wheel 204. In an alternative variant, shown in FIGS. 18a and 18b, the primary screw 205 is a notched bar, engaged internally with the primary wheel 204. In both of the above examples, depending on the direction of rotation of the primary wheel 204, it is possible to move the primary screw 205 longitudinally (forward and backward), which, by moving, acts on the tie rod 202 thus allowing for the curvature of the distal portion of the guide rod 2.

The guide stem 2 comprises an outer body 22, which is rigid and slidable over the inner body 21, suitable for adjusting the length of the flexible part of the guide stem 2, thus also acting on the curvature of the distal portion. In fact, as shown in FIGS. 6a and 6b, depending on the level of advancement of the outer body 22 on the inner body 21, the flexible distal portion of the guide stem 2 will assume different curvatures, either narrower (FIG. 6b—outer body 22 advanced) or wider (FIG. 6a—outer body 22 retracted).

The outer body 22 is rigid, made, for example, of metal or rigid plastic.

In an embodiment example, shown in FIGS. 20 and 21, the distal end portion of the outer body 22 has a flexible section 44 obtained by creating a series of openings 445 in the outer body 22. These openings 445 are preferably aligned and parallel, and are interspersed with ribs 444, i.e. non-removed portions of the outer body 22.

Preferably, the flexible section 44 of the outer body 22 is made of the same material as the outer body 22, or it is an insert applied to the outer body 22.

The distal portion 103 of the grip 1 houses secondary curvature means 60 of the guide stem 2 connected to the outer body 22 to control its advancement and retraction according to the desired degree of curvature of the flexible distal portion of the guide stem 2.

The secondary curvature means 60 comprise a secondary wheel 601 connected to a secondary worm screw 602 with which the outer body 22 is integral. According to the direction of rotation of the secondary wheel 601, it is possible to move the outer body 22 longitudinally (forward and backward), which by moving, acts, freeing or forcing into position, the flexible distal portion of the guide stem 2.

With reference to FIGS. 6a and 6b:
configuration A, flexible distal segment of the guide stem 2 in the rest position;
configuration B, activation of the tie rod 202, with a greater arching of the flexible distal portion of the guide stem 2 in FIG. 6 a, where the outer body 22 is retracted, with respect to FIG. 6b, where the outer body 22 is advanced.

In the embodiment shown for example in FIGS. 3 and 12, the secondary screw 602 is a notched cylinder and is engaged internally with the secondary wheel 601. In an alternative embodiment, shown in FIGS. 18a and 18b, the secondary screw 602 is a notched bar, engaged internally with the secondary wheel 601. In both solutions described above, according to the direction of rotation of the secondary wheel 601, it is possible to move the outer body 22 longitudinally (forward and backward), which, by moving, acts, freeing or forcing into position, the distal flexible portion of the guide stem 2.

In one embodiment, depicted in FIGS. 18a and 18b, both the primary screw 205 and the secondary screw 602 are in the form of a notched bar. Preferably, the two bars are arranged in distinct planes, preferably parallel or perpendicular to each other. Preferably, the projections of the bars along the longitudinal axis of the device are superimposed axially in such a way as to reduce the overall dimensions to the minimum without obstructing each other as they slide along their main direction of development.

Optionally, the primary operating means 203 comprise a visual reference indicator 206, which moves longitudinally (forward and backward) integrally with the primary screw 205 to indicate the state of advancement of the outer body 22.

The dilation balloon 3 is shown in detail in FIGS. 9 and 10.

The dilation balloon 3 is integrated into the multifunctional device 100 and is not removable therefrom.

The dilation balloon 3 comprises a balloon 31 mounted on a support tube 32 provided with an inflation lumen 321 connected on one side to relevant inflation/deflation means 80 and flowing, on the opposite side, inside the balloon 31.

The support tube 32 ends in a tip 33 designed to be atraumatic during navigation into the site to be treated.

According to an embodiment of the present invention, shown in FIG. 22, the tip 33 of the support tube 32 is provided with an electrode 333.

Preferably, the support tube 32 is provided with a reinforcing sheath 329, integral therewith, that is rigid, e.g., made of metal, suitable for facilitating the thrust of the dilation balloon 3.

The support tube 32 is also provided with an additional lumen 322 connected on one side to suction/injection means 70 and leading, on the opposite side, outwards through the tip 33.

The dilation balloon 3 is slidingly inserted into the first inner channel 223 of the guide stem 2 and is guided in advancement and retraction by position adjustment means 30.

The position adjustment means 30 comprise a tertiary wheel 301 connected to a tertiary worm screw 302 with which the support tube 32 is integral. According to the direction of rotation of the tertiary wheel 301, it is possible to move the dilation balloon 3 longitudinally (forward and backward) to cause it to exit and re-enter from the tip 23 of the guide stem 2. The dilation balloon 3, and in particular the support tube 32 provided with two lumens 322, 321 is connected integrally to the tertiary screw 302. In particular, the support tube 32 passes longitudinally through the tertiary screw 302 internally, as seen in FIG. 9.

As seen in FIG. 12, the dilation balloon 3, and in particular the support tube 32 provided with two lumens 322, 321, also passes longitudinally through the primary screw 205 of the primary operating means 203 internally, without being connected or integral therewith. Finally, as seen in FIG. 2, the dilation balloon 3, and in particular the support tube 32 provided with two lumens 322, 321, also passes longitudinally through the secondary screw 602 of the secondary curvature means 60 internally, without being connected or integral therewith.

In the embodiment, shown for example in FIGS. 3 and 12, the tertiary screw 302 is a notched cylinder, and is engaged internally with the tertiary wheel 301. In an alternative embodiment, shown in FIGS. 18a and 18b, the tertiary screw 302 is a notched bar, engaged internally with the tertiary wheel 301. In both such solutions, depending on the direction of rotation of the tertiary wheel 301, it is possible to move the dilation balloon 3 longitudinally (forward and backward) to cause it to exit and re-enter from the tip 23 of the guide stem 2.

In one embodiment, depicted in FIGS. 18a and 18b, all the worm screws (primary 205, secondary 602, and tertiary 302) have the form of notched bars. Preferably, the three bars are arranged in distinct planes, preferably parallel and/or perpendicular to each other. Preferably, the projections of the bars along the longitudinal axis of the device are superimposed axially in such a way as to reduce the overall dimensions to the minimum without obstructing each other as they slide along their main direction of development. Advantageously, therefore, as seen in the embodiment shown in FIG. 20, the primary operating means 203, the position adjustment means 30, and the secondary curvature means 60 are all contained in the grip 1. The worm screws, all in notched bar form, engage the respective wheels internally, and are arranged in distinct planes (parallel and/or orthogonal) so that they do not obstruct each other when sliding.

In the embodiment in FIG. 19, the support tube 32 passes longitudinally through all the wheels, primary 204, secondary 601, and tertiary 301, internally.

Optionally, the position adjustment means 30 comprise a visual reference indicator 303, which moves longitudinally (forward and backward) integrally with the dilation balloon 3 to show its state of advancement.

It should be noted that when a position indicator 303, 206 is present, the grip 1 is provided with a relative opening 903, 906 suitable for displaying the sliding of the position indicator 303, 206.

As mentioned above, the dilation balloon 3 is inflated by relevant inflation/deflation means 80. FIG. 9 shows the balloon 31 in a deflated configuration, and FIG. 10 shows the balloon 31 in an inflated, i.e., expanded, configuration.

The inflation/deflation means 80, clearly visible in FIG. 2, comprise a syringe 81 provided with a piston 82 and, preferably, a safety valve 83. The inflation/deflation means 80 are removable from the multifunctional device 100. At least the syringe 81 is removable so it may be loaded. As seen in FIGS. 2, 4, and 5, the grip 1 comprises a syringe housing 111, suitable to house indeed the syringe 81.

According to a further embodiment, shown in FIG. 20, the inflation lumen 321 terminates in a connector 111 connected to the inflation/deflation syringe 81 by an extension 112. Such solution allows for easy and independent movement of the syringe 81 connected to the multifunctional device 100 by means of the extension 112.

The safety valve 83 is properly calibrated so that the balloon 31 does not reach critical burst situations. The safety valve 83 is provided with a syringe inlet 831, connected to the syringe 81, and a balloon outlet 832 connected to the inflation lumen 321. The safety valve 83 also comprises a valve body 833 of the spring-loaded piston type and a vent 834 at the bottom. When the pressure delivered by the syringe 81 exceeds a safety pressure, the valve body 833 is activated: the pressure generated by the syringe overcomes the force of the spring, and the piston lowers, opening a passage to the vent hole at the bottom of the safety valve.

Preferably, the syringe 81 is provided with a selector 84 that allows for activating, FIG. 13a, and deactivating, FIG. 13b, controlled advancement means 841 of the piston 82. When the controlled advancement means 841 are deactivated, as in FIG. 13 a in which the Serration of the selector 84 is disengaged from the serration of the piston 82, the advancement of said piston is of the free type, i.e., slidable by thrust. Such free typology is usually used for loading the syringe 81. When the controlled advancement means 841 are activated, as in FIG. 14a in which the serration of the selector 84 engages the serration of the piston 82, the movement of the piston 81 is gradual, by screwing, thus allowing for a progressive and partial inflation of the balloon 31.

For example, the controlled advancement means 841 are of the screw serration type. In such an example, the piston 82 is at least partially threaded, for example with a trapezoid thread, so as to ensure a good seal with the female thread made on the selector 84. Furthermore, the piston 82 terminates proximally with a piston wheel 842 for the micrometric movement of the piston. The piston wheel 842 facilitates the advancement by rotation when the controlled advancement means 841 are activated.

As mentioned above, the support tube 32 is also provided with an additional lumen 322 for suction/injection means 70 that indeed allow for either injecting a fluid or suctioning from the tip 33 of the dilation balloon 3. The suction/injection means comprise a connector 71 directly connected to the additional lumen 322 and integrated with the multifunctional device 100, i.e., non-removable therefrom. Further injection and/or suction devices, not shown, such as a syringe, may be connected to such connector 71.

In the embodiment example in FIGS. 20 and 22, the additional lumen 322 of the support tube 32 is connected to an electrified cable 382 suitable for activating an electrode 333 housed on the tip 33 of the support tube 32 of the dilation balloon 3.

As mentioned above, the device 100 further comprises a connector 4 for the insertion of further devices that may be used according to specific needs. The connector 4 is directly connected to the second internal channel 224 that exits from the tip 23 of the guide stem 2.

In one embodiment example, shown in FIGS. 2 and 15, the further device 90 is a radiofrequency catheter.

The radiofrequency catheter comprises a knob 91 connected to a catheter body 92 at the distal end of which the accessories 93, in this case the electrodes, are arranged. These electrodes, indicated in the figures by “+” and “−”, allow, when activated, to obtain a precise thermostatic ablation in the desired site.

The knob 91 comprises a knob wheel 94 that allows, by turning, the advancement and retraction of the catheter body 92 with respect to said knob. Therefore, when the radiofrequency catheter is connected to the multifunctional device 100, i.e., when the catheter body 92 is inserted into the second inner channel 224 of the guide stem 2, and the knob 91 is engaged to the connector 4, the rotation of the knob wheel 92 advances the catheter body 92 until it exits at least partially from the tip 23 of the guide stem 2 and until the electrodes 93 are correctly positioned in the area to be treated. The radiofrequency catheter 9 is of the bipolar radiofrequency with thermocouple type to perform thermostatically controlled microablations.

In a further embodiment example, the further catheter 90 is a drill catheter (driller). In such example, the drill catheter comprises a knob 91, which is connected to a catheter body 92 at the distal end of which the accessories 93, in this case the drill bit, are arranged. The advancement operation is exactly the same as described for the radiofrequency catheter.

It should be noted that the presence or absence of the further catheter 90 is an additional function that the user may decide to use or not use. In fact, in contrast to the dilation balloon 3 that is already integrated in the multifunctional device 100, the further catheter 90 is an accessory, which may be mounted on or removed from the connector 4 as needed, at the discretion of the practitioner and according to the pathology encountered.

The multifunctional device 100 object of the present invention enables the balloon sinuplasty technique to be performed without risk, on all patients, even in conjunction with ongoing nasal or general medical therapies for said sinusitis. By virtue of the multifunctional device 100, the otorhinolaryngologist performs dilation of the natural openings of the paranasal sinuses through pressure inflation the balloon 31, tailoring the treatment to the patient's anatomical and clinical condition. The multifunctional device 100, with its variable-angle distal portion, allows for reaching any type of anatomical region involved in the patient's pathology. The multifunctional device 100 integrates the dilation balloon, which, once inflated to the intended pressure, allows for atraumatic drainage of the sinusitis without bleeding or pain, while preserving all of the surrounding nasal anatomy.

With regard to the otofunctional results achievable with tubal dilation treatment using the multifunctional device 100, a rapid recovery of the proper ventilation of the middle ear is obtained, with liberation from tympanic catarrh stagnation, reduction of submucosal inflammation of the tubal lining, and resolution of compensation difficulties at altitude changes or underwater activities. It is particularly advantageous to fast train or air travelers (frequent flyers) and those who suffer from recurrent colds with catarrhal involvement of the ear.

Innovatively, a multifunctional device with an integrated dilation balloon according to the present invention is provided with a series of elements all suitably integrated, with a minimal size, so that the user holds the entire assembly in one hand and with thumb and forefinger is able to operate the various movement and control means that enable its use.

Advantageously, the device 100 comprises a directly integrated and non-removable dilation balloon 2.

Advantageously, the device 100 comprises a directly integrated connector 4 for the insertion additional deployable devices according to specific needs.

Advantageously, the device 100 comprises directly integrated control means (such as, for example, means for adjusting the curvature of the guide stem 2, means for adjusting the position of the dilation balloon 3, means for inflating/deflating the dilation balloon 3, and suction/injection means) that are space-saving and all operable with only one hand.

Advantageously, when the worm screw of the control means integrated in the device is in the form 44 a notched bar engaged within the respective wheel, it is possible to minimize the size and reduce the weight of the device 100, further improving its ergonomics and maneuverability.

Advantageously, the means for adjusting the curvature of the guide stem 2, and/or the means for adjusting the position of the dilation balloon 3, are of the wheel type connected to a worm screw, which is easy and precise to operate with only one hand.

Advantageously, the multifunctional device 100 is easy to use because the various means integrated therein are operable with only one hand. That is, the inflation/deflation means 80 of the dilation balloon 3, the suction/injection means 70, the primary curvature means 20 of the guide stem 2, the secondary curvature means 60 of the guide stem 2, the position adjustment means 30 of the dilation balloon 30, and, when present, the further device 90, are all operable with only one hand.

Advantageously, the dilation balloon 3 passes longitudinally through the curvature adjustment means of the guide stem 2 and/or the position adjustment means of the dilation balloon 3 internally, thus allowing for the size to be minimized.

Advantageously, the worm screw of the various control and adjusting g means, when in the form of a notched cylinder, is hollow internally to allow the passage of the dilation balloon 3, thus allowing the overall dimensions to be reduced to a minimum.

According to an additional advantage, the outer body is provided with a flexible section, obtained through a series of openings, which provides more flexibility to the guide stem, improving the maneuverability of the device.

It is clear that those skilled in the art may make changes to the device described above in order to meet incidental needs, all falling within the scope of protection defined in the following claims.

1. A multifunctional medical device with an integrated dilation balloon, comprising:
a grip connected to a guide stem;
a dilation balloon slidingly inserted into the grip and the guide stem and not removable therefrom;
means for adjusting a curvature of the guide stem, associated with the grip and/or the guide stem and not removable therefrom;
position adjustment means for adjusting a position of the dilation balloon, associated with the grip and not removable therefrom; and
inflation/deflation means for inflating/deflating the dilation balloon, associated or associable with the grip.

2. The multifunctional medical device of claim 1, further comprising a connector for insertion of further medical devices, the connector being fixed to the grip and not removable therefrom.

3. The multifunctional medical device of claim 1, further comprising suction/injection means.

4. The multifunctional medical device of claim 2, wherein the guide stem comprises an inner body fixed to the grip, provided with a first inner channel, in which the dilation balloon is slidingly housed, and a second inner channel connected to the connector.

5. The multifunctional medical device of claim 1, wherein the guide stem comprises:
an inner body with a flexible distal portion capable of switching from a straight configuration to a curved configuration through the means for adjusting the curvature of the guide stem, and wherein the means for adjusting the curvature of the guide stem are primary curvature means and comprise:
at least one tie rod housed in the inner body; and
primary operating means in a form of a primary wheel connected to a primary worm screw, with which the at least one tie rod is integral.

6. The multifunctional medical device of claim 1, wherein the guide stem comprises:
an inner body with a flexible distal portion capable of switching from a straight configuration to a curved configuration through the means for adjusting the curvature of the guide stem, and wherein the means for adjusting the curvature of the guide stem are secondary curvature means and comprise:
an outer rigid body sliding above the inner body; and
a secondary wheel connected to a secondary worm screw with which the outer rigid body is integral.

7. The multifunctional medical device of claim 6, wherein the outer rigid body is distally provided with a flexible section comprising a plurality of openings interspersed with ribs.

8. The multifunctional medical device of claim 1, wherein the dilation balloon comprises a balloon mounted to a support tube, the support tube being provided with a tip and an inflation lumen connected on one side to the inflation/deflation means and flowing, on an opposite side, inside the balloon.

9. The multifunctional medical device of claim 8, wherein the support tube is also provided with an additional lumen connected on one side to suction/injection means and flowing, on an opposite side, outwards through the tip.

10. The multifunctional medical device of claim 8, wherein the position adjustment means comprise a tertiary wheel connected to a tertiary worm screw with which the support tube of the dilation balloon is integral.

11. The multifunctional medical device of claim 10, wherein the dilation balloon longitudinally crosses internally the tertiary worm screw and/or the tertiary wheel.

12. The multifunctional medical device of claim 1, wherein the dilation balloon is slides inside the means for adjusting the guide stem curvature of the guide stem and/or the position adjustment means.

13. The multifunctional medical device of claim 5, wherein the primary worm screw is cylindrical or is a notched bar.

14. The multifunctional medical device of claim 1, wherein the inflation/deflation means of the dilation balloon comprise:
a syringe provided with a piston;
a safety valve provided with a vent; and
a selector which activates and deactivates controlled advancement means of the piston.

15. The multifunctional medical device of claim 1, further comprising a further catheter provided with a knob connected to a catheter body at a distal end of which accessories are arranged, and wherein the catheter body is inserted into a second inner channel of the guide stem and the knob is engaged with a connector.

16. The multifunctional medical device of claim 6, wherein the secondary worm screw is cylindrical or is a notched bar.

17. The multifunctional medical device of claim 10, wherein the tertiary worm screw is cylindrical or is a notched bar.
